(19) United States
(12) Patent Application Publication
(10) Pub. No.: US 2008/0081965 A1
(43) Pub. Date: Apr. 3, 2008

(54) FOREIGN BODY RESPONSE DETECTION IN AN IMPLANTED DEVICE

(75) Inventors: Carl F. Edman, San Diego, CA (US); Darrel D. Drinan, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PhiloMetron, Inc., San Diego, CA (US)

(21) Appl. No.: 11/862,069

(22) Filed: Sep. 26, 2007

Related U.S. Application Data
(60) Provisional application No. 60/848,345, filed on Sep. 29, 2006.

(51) Int. Cl. A61B 5/00

(52) U.S. Cl. 600/309; 623/1.13

(57) ABSTRACT

A medical device with an implantable portion comprises, in part, an encapsulation sensor. In preferred embodiments, the encapsulation sensor comprises at least two electrodes and a circuit configured to sense impedance between the electrodes. Cell accumulation and fibrous capsule growth causes an increase in impedance. Functionality of the sensor can be evaluated based at least in part on the sensed impedance.

### CROSS REFERENCE TO RELATED APPLICATIONS

This application claims priority under 35 U.S.C. Section 119(e) to provisional application No. 60/848,345, filed on Sep. 29, 2006.

### BACKGROUND OF THE INVENTION

A limiting factor in maintaining adequate, optimal or intended functions of devices implanted within the body of a mammalian subject is the body's rejection of or reaction to these materials, termed the “foreign body response”. In this context, the foreign body response includes any or all of those events initiated by the body in reaction to introduced material. This includes, but is not limited to, inflammation response, migration of macrophages or other wound/repair cells to the location, altered cell type of the surrounding tissue, deposition of fibrous proteins and related materials not normally observed within the particular tissue in those forms or levels, and/or the walling off or encapsulation of the device by the body by a fibrous capsule.

Many devices include analyte sensors and/or drug delivery ports that are adversely affected by this encapsulation. In some cases, the analyte sensor retains functionality, but its output is not accurate. Detecting the presence of encapsulation would improve the ability to distinguish between true changes in analyte concentrations and encapsulation caused changes to sensor readings. Likewise, encapsulation surrounding a delivery port may retard or prevent transmission of the delivered agent to surrounding tissue.

A foreign body response may also occur as the result of a device or materials placed within the vasculature. In this context, the foreign body response may include a build-up of cellular materials, termed a stenotic response, in the region of the implanted device or materials. This stenotic response may lead to the occlusion of the vessel, and potentially, to thrombosis.

### SUMMARY OF THE INVENTION

Encapsulation detection in the context of the invention represents the detection of the body's foreign body response to an implanted medical device or the measurement of the foreign body response in general. Thus, the term “foreign body response” may also in certain circumstances represent the body's response to certain disease states or conditions wherein the body's reaction to a disease state in a particular tissue or body structure resembles that response presented to a foreign material or introduced medical device. In certain aspects of the invention, the foreign body response may also arise from transplanted organs or other biological materials, rather than manufactured devices, structures or substances. The scope of the invention is therefore not limited to any one underlying cause for foreign body reaction or location within the body.

The sensor may comprise a component of the implanted medical device or may represent a separate component so positioned as to be able to evaluate foreign body formation on or near the implanted medical device or region of the foreign body response.

In one embodiment, the invention comprises a method of determining analyte concentration by an implanted device. The method comprises sensing at least one analyte concentration with an analyte sensor in the implanted device, sensing encapsulation of the implanted device while the implanted device remains implanted, and evaluating accuracy of sensed analyte concentration based at least in part on the presence or absence of sensed encapsulation.

In another embodiment, the invention comprises a medical device having an implantable portion. The medical device comprises at least one of an analyte sensor and a fluid delivery port in the implantable portion and an encapsulation sensor. The encapsulation sensor may comprise an impedance sensor.

In yet another embodiment, a medical device having an implantable portion comprises at least one of an analyte sensor and/or a fluid delivery port in the implantable portion, at least one electrode positioned proximate to the analyte sensor and/or fluid delivery port, and at least one additional electrode. In addition, an electrical signal generator is connected across at least two of the electrodes and is configured to cause current to pass between the at least two electrodes. Also provided is a sensing circuit configured to measure electrical impedance between the at least two electrodes.

In an alternate embodiment of the invention, the sensor may monitor foreign body formation about a region or aspect of an implanted medical device, e.g. an implanted stent or graft, whose primary function does not involve analyte sensing, such as a vascular graft.

In another embodiment, a method of treating a subject with an implanted medical device comprises detecting encapsulation of the implanted device while the implanted device remains implanted, and replacing the implanted device when adverse encapsulation is detected.

In one advantageous embodiment, sensing encapsulation comprises sensing impedance between a pair of electrodes. In alternate embodiments, the sensing methodology comprises the exchange of at least one form of energy between the sensor and the tissue such that the degree of foreign body formation may be determined. Such forms of energy may include, but are not limited to, electrical energy such as electric impedance, electromagnetic energy (radiowaves of one or more frequencies), acoustic energy, mechanical energy or optical photonic) energy. Upon receipt of information regarding foreign body formation, clinical intervention may therefore be taken to relieve the foreign body build-up and/or medical condition underlying the foreign body response.

### BRIEF DESCRIPTION OF THE DRAWINGS

FIG. 1 is a block diagram of an implanted system incorporating aspects of the invention;

FIG. 2 is a diagram of an implanted portion of a medical device in accordance with an embodiment of the invention;

FIG. 3 is a diagram of a fully implanted embodiment of the invention.

FIG. 4 is a diagram of an implanted vascular graft with associated sensor as an implanted medical device in accordance with an embodiment of the invention.

FIGS. 5A and 5B show a representation of an electric impedance sensor with a vascular graft in an embodiment of the invention.

FIGS. 6A and 6B show another implanted vascular graft having a sensor in accordance with an embodiment of the invention.

### DETAILED DESCRIPTION OF THE PREFERRED EMBODIMENT

The following detailed description is directed to certain specific embodiments of the invention. However, the invention can be embodied in a multitude of different ways. In this description, reference is made to the drawings wherein like parts are designated with like numerals throughout.

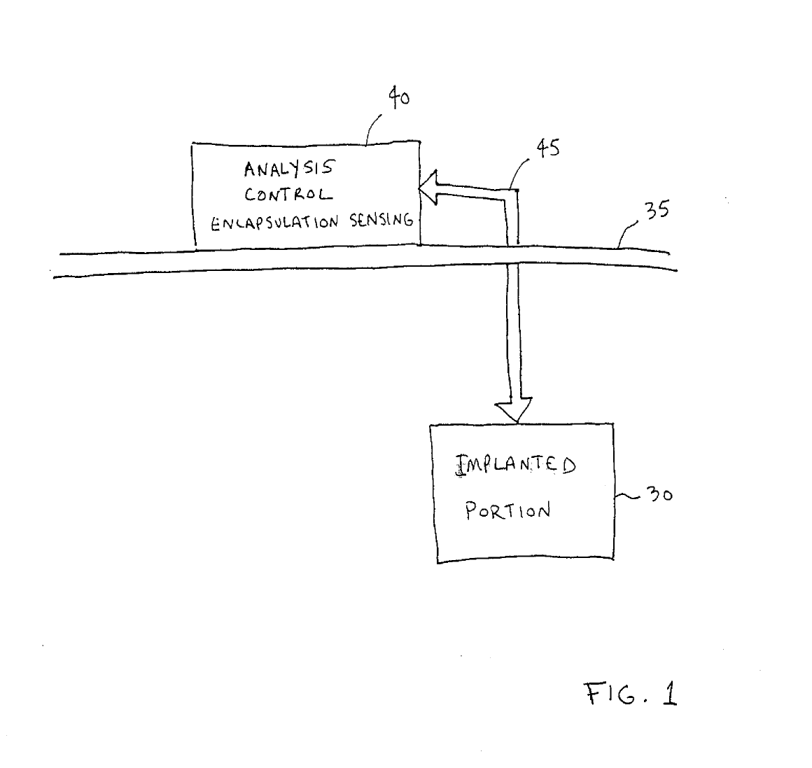

FIG. 1 illustrates one embodiment of the invention. The medical device in this embodiment comprises two portions. The first is an implanted portion 30 which is placed in the body of the subject under the skin 35. The implanted portion 30 will typically include an analyte sensing mechanism and/or a drug delivery port. The analyte sensor can be chemical, optical, MEMS based, or any of a wide variety of available technologies. The analyte or analytes detected may also vary widely and may include glucose, circulating hormone levels, concentrations of administered therapeutic agents, etc. The implanted portion 30 is in communication with a control unit 40 via a communication interface 45. The control unit 40 may include a power source such as a battery as well as processing and logic circuitry for controlling circuits in the implanted portion and for analyzing data received from the implanted portion. The communication path 45 may be wired and extend through the skin or may be wireless. In some embodiments, the control unit 40 includes a reservoir of therapeutic material and the communication path 45 comprises tubing or other fluid communication mechanism for delivering the therapeutic material to the implanted portion to the subject through a fluid delivery port. The control unit 40 can reside in a variety of locations. It can be mounted on the subject or be stationary in the vicinity of the subject. It will be appreciated that in some embodiments, the control unit 40 can also be implanted and/or combined with the implanted portion 30 as a single implanted unit.

In accordance with one aspect of the invention, the control unit further comprises an encapsulation detector. This detector is configured to detect cell accumulation, fibrous capsule formation, and other material that accumulates on or near the device due to the subject's foreign body response. With such a detector, the accuracy of any data received from an analyte sensor can be evaluated. If no encapsulation is detected the analyte sensor is likely accurate. However, if encapsulation is detected, the output becomes suspect. Advantageously, the encapsulation detection can be performed while the device remains implanted. This is useful because the rate of device encapsulation proceeds at very different rates devices implanted at different times or different places and in different subjects. Currently, implanted devices must be removed and replaced in a short period of time that guarantees no adverse effects from encapsulation over the complete range of subjects and treatments. However, an implanted device that is removed and replaced weekly may sometimes last a month or two or even longer if given the chance. With the invention, devices that resist encapsulation longer can remain in the body longer, thus increasing the average time span between device replacement.

In some advantageous embodiments, the encapsulation sensor comprises an electrical impedance sensor. In this embodiment, and as explained further below, electrodes are placed such that the growth of encapsulation impedes an electrical current between the electrodes. An increase in impedance detected between the electrodes is an indication of encapsulation of the device. In the context of this invention, the term “electrodes” is not limited to metallic or semimetallic conductive structures but may consist of a variety of conductive or semiconductive materials in various geometries not limited to those described herein.

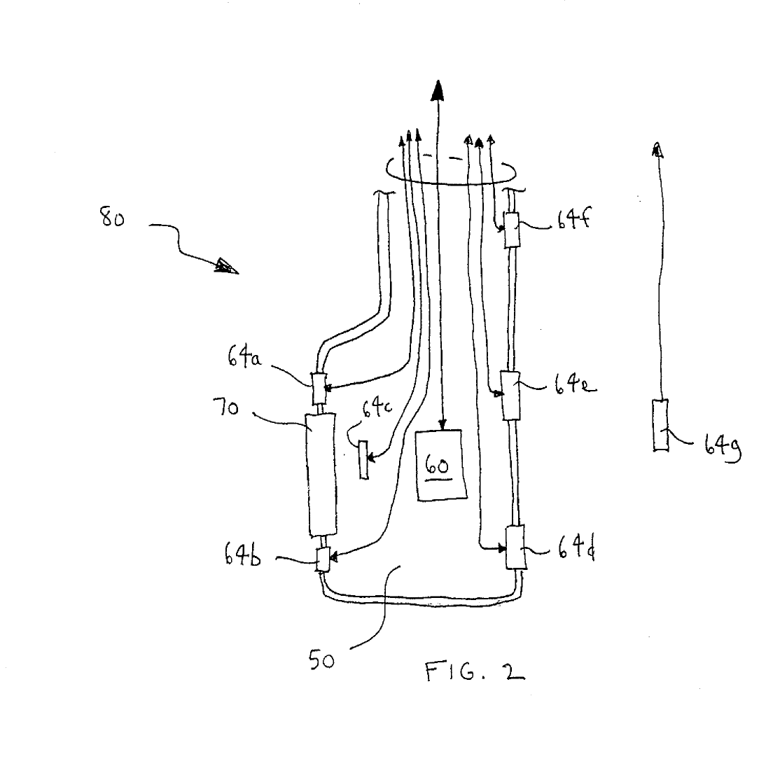

In FIG. 2, one end of an analyte sensing and/or fluid delivery device 80, e.g. a catheter like device, is shown having a luminal space 50. A semipermeable structure 70 may serve as the site of fluid delivery from the interior luminal space of the device. Fluid passing down the luminal space of the catheter can exit from the device 80 through the semipermeable structure 70 and pass into the surrounding tissue. Interstitial or other body fluids can also enter into the luminal space 50 via the semipermeable structure 70. Positioned within this luminal space and beneath the semipermeable structure 70 may be an analyte sensor 60. This sensor 60 is connected to a power supply/control unit 40. The device 80 further includes at least two electrodes, designated 64a-g in FIG. 2. Upon activation of the electrodes, the electrical current passes from the surface of one or more of the electrodes into body fluids, traverses through the fluid and/or tissue and completes the circuit at another one or more electrodes. It should be noted that no orientation or polarity of activation is implied by this description of the electrical pathway.

The multiple electrodes in FIG. 2 may be in any number and any arrangement. Generally two or more will be provided, with at least one typically mounted proximate to the fluid delivery port 70 and/or the analyte sensor 60. One or more may be inside the luminal space 50. It may be advantageous to provide multiple electrodes such as shown in FIG. 2 and force current flow between respective pairs of electrodes at different times to perform comparisons. As shown by electrode 64g, one or more electrodes could be placed off the device adjacent to the implant site or even on the external surface of the subject's body.

The electrodes are connected to the power and control unit 40 via wires. The control unit 40 may include either or both a voltage source and current source. The impedance between the electrodes can be determined by measuring the current produced at a given voltage or the voltage required to produce a given current. Resistive and capacitive components can be resolved with current-voltage phase measurements of AC waveforms. Frequency can be fixed or varied. Encapsulation, e.g. deposited collagen and cells associated with this deposition, has been shown to produce significant impedance changes to applied AC voltages in the 10 KHz to 100 KHz range (see, for example, Warren M. Grill and J. Thomas Mortimer, Electrical Properties of Implant Encapsulation Tissue, Ann. Biomed. Eng. 22: 23-33 1994), although a wide variety of frequencies, and even DC, could be used. Because encapsulation occurs over along period of time, application of voltages to the electrodes need only be intermittent, avoiding electrolysis byproducts and detrimental pH changes near the electrodes.

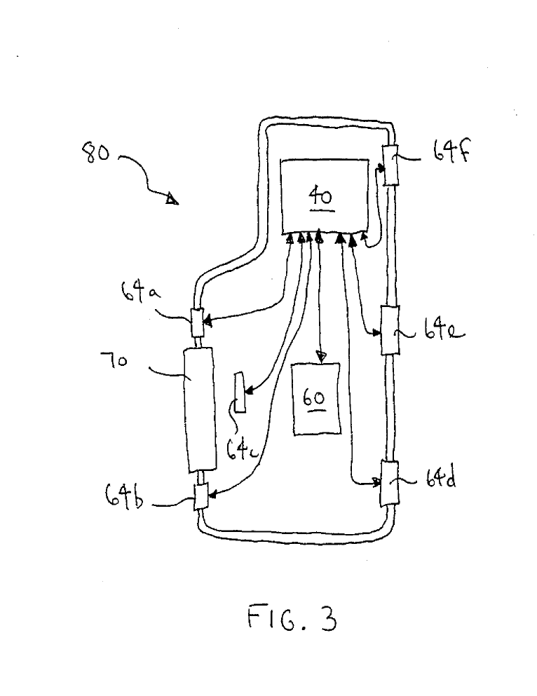

FIG. 3 is a diagram of a fully implanted device having the power and control unit 40 incorporated into the implant.

An impedance based system of encapsulation detection can be combined with the electrophoresis based encapsulation minimization techniques described in US Patent Publication Number 2004/0106951, the disclosure of which is hereby incorporated by reference in its entirety. The electrodes described in this publication could be used to both control cell migration and detect encapsulation.

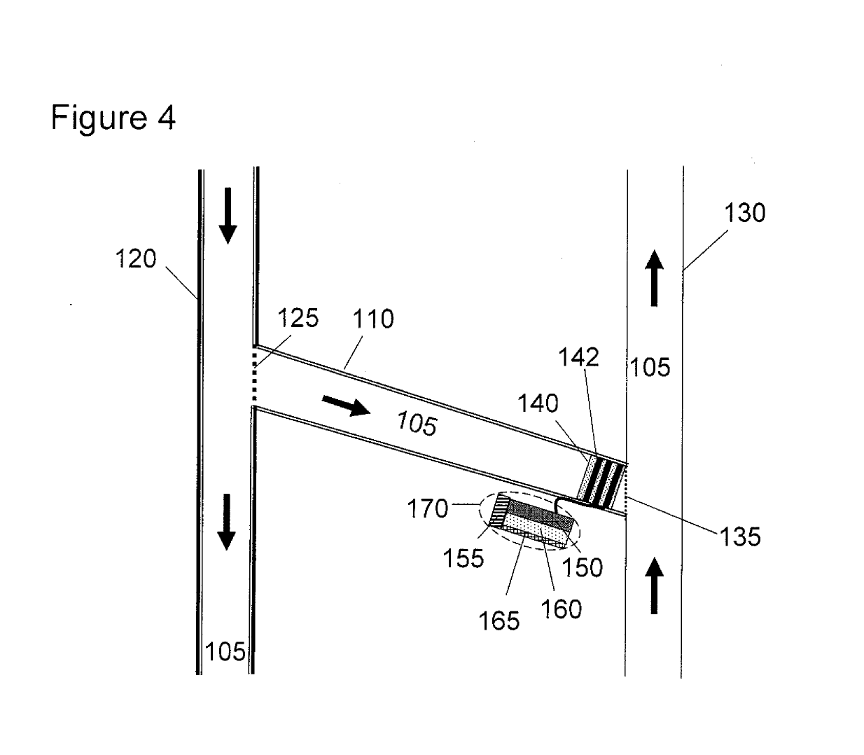

Another embodiment of the invention is shown in FIG. 4. Medical device 110 comprises a vascular graft 110 positioned between an artery 120 and vein 130 with anastomoses indicated by 125 and 135, respectively. Predominant blood flow through this region is shown by arrows located within the lumenal space 105 of the vessels and device. In this embodiment, sensor 140 is shown positioned in the vicinity of the venous anastomosis 135. Sensor 140 is connected to impedance detection circuitry 150 positioned in the vicinity of the medical device via wires. Several electrodes 142 comprising active components of sensor 140 within this embodiment of the invention are positioned within the lumenal space of the medical device 110 and in advantageous fashion comprise part of the structure of the medical device. In general, electrodes may be in any number and any arrangement within the scope of this invention, located both inside and/or outside the luminal spaces.

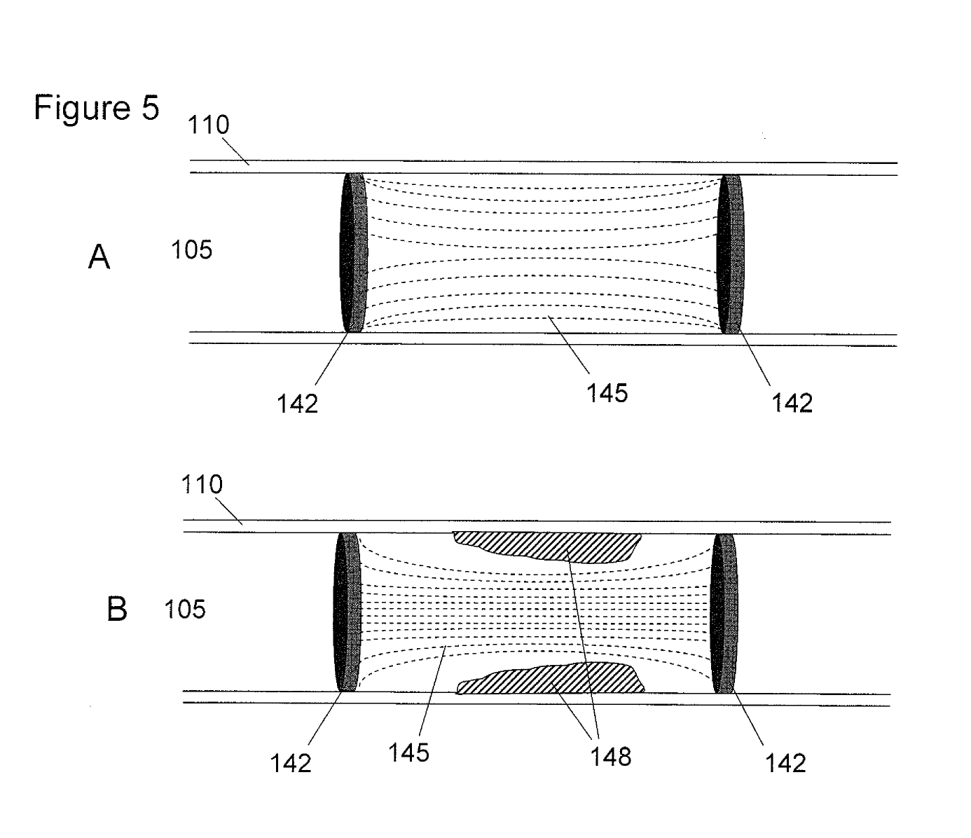

The passage of the electrical current from one electrode to the other electrode is affected by passage through the stenotic material resultant from the body's foreign body response to the introduced device. FIGS. 5A and 5B represents the conceptual flow of electrical current 145 through lumenal space 105 between concentric ring electrodes 142 within device 110. FIG. 5A illustrates electrical flow in the absence of stenotic build-up within said lumenal space and FIG. 5B illustrates electrical flow being impeded by stenotic build-up 148. Stenotic build-up is believed to present significantly greater resistance to electrical flow than blood within a constrained volume presented by the structure of the device. Under such conditions, a change in electrical impedance may then be registered and attributable to formation of the stenotic build-up.

In general application of the invention in this embodiment, measurement of the foreign body response, e.g. stenotic build-up on the lumenal aspect of the graft, may be ascertained by comparative measurements taken periodically over an extended period of time, e.g. days, weeks or months, for the determination of change of impedance associated with the presence of hyperplasia, fibrous material or other attributes of stenosis arising from the introduced medical device. Such measurements take advantage of the high conductivity of blood as compared to tissue such that increases in impedance attributable to tissue/fibrous material growth are readily determined. Such measurements may require additional methods to remove non-specific signals not attributable to tissue growth per se. Such signals may arise from pulsality of the blood flow, general body movement and/or change in hematocrit concentration over time. Removal of this unwanted signal noise may be accomplished by signal averaging of multiple measurements, selection of measurement periods during periods of minimal body motion, e.g. during sleep, or by combining measurements with one or more physiological measurements taken by one or more other medical devices, e.g. blood sample analysis, weight change indicating hydration status, etc. The method of signal noise analysis is not constrained by any one form of analysis or sensor input.

Returning to FIG. 4, power for impedance measurements and other electrical circuitry functions is provided by power source 155. In preferential embodiments of the invention, such power is supplied by long lasting batteries, however, the method and devices of this invention are not constrained to any one form or type of power source. Alternative forms of power, e.g. power sources arising from externally electromagnetic coupling, may also be employed to enable the invention.

Communication of sensor data or processed forms of sensor data may be accomplished by transmission circuitry 160 with antenna 165 electrically connected to impedance circuitry 150. In such embodiments, a preferred form of communication utilizes the Medical Implant Communications Service (MICS) radio wave band, 402 MHz to 405 MHz, to enable common communication with other clinic devices and services. Alternate forms of communication are conceivable, e.g. other radio frequencies, acoustics, or optical, to transmit data between the sensor and the exterior of the body, and are well known to those familiar with the art of implanted electronic devices. The scope of this invention is not restricted to any one form or method of communication.

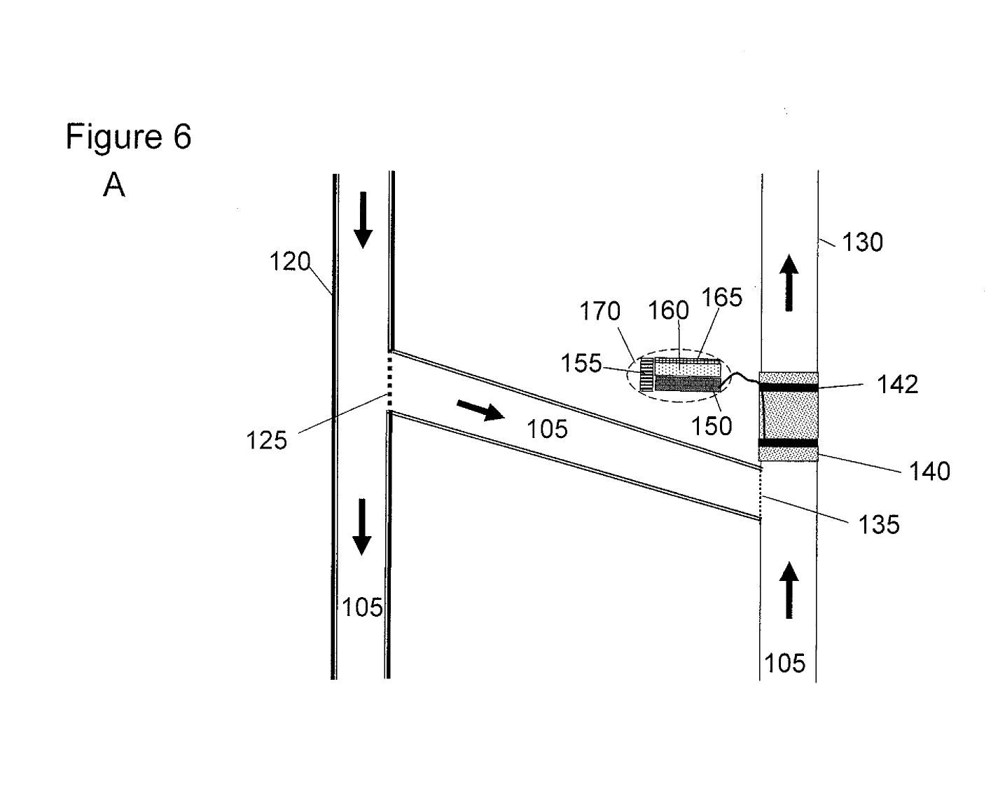

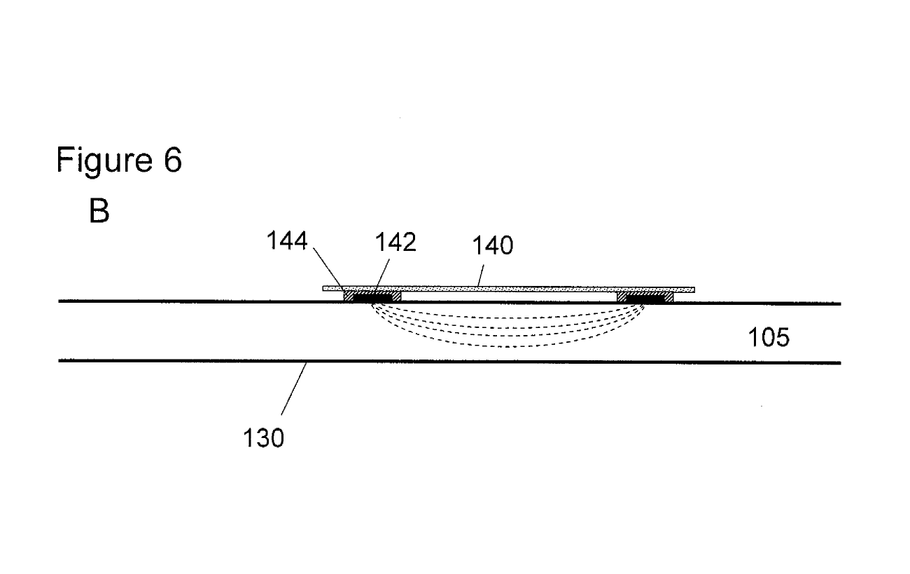

In a variation of the above described embodiment of the invention, FIG. 6A indicates the position of sensor 140 located on the exterior surface of vein 130 adjacent to anastomosis 135 and separate from medical device 110. In such embodiments, foreign body response not may not arise directly on medical device 110 but results from the introduction of said device into the body. Specifically, in arteriovenous grafts, the altered blood flow and pressures arising from graft introduction are believed to promote formation of stenosis in venous regions in the vicinity of the anastomosis. To facilitate this determination, an insulating layer 144, FIG. 6B, may be positioned on at least one electrode surface to orient impedance measurement preferentially through the vessel lumenal space 105.

In the embodiments of invention for detection of foreign body response such as stenosis presented in FIG. 4 and FIG. 6, the stenosis may be detected directly at the site of the electrodes or intervening region between one or more sets of electrodes by the change electrical impedance resultant from the reduction in cross sectional area of the relatively highly conductive blood as compared to the less conductive vessel wall and stenosis formation.

Alternative forms of sensor 140 may be employed for foreign body response detection in these and other embodiments of the invention. Such sensors may be electromagnetic, radiowave, optical, acoustic or mechanical in nature. For example, implanted sensors utilizing one or more sonic transmitters and receivers may be positioned about one or more vascular structures to evaluate progressive change in vessel wall thickness or compliance. Change in wall thickness or compliance may result in change of transmitted signal thereby indicating a change in vessel structural characteristics, e.g. thickening, over time. This approach is distinct from other sonic approaches such as ultrasonic monitoring or phonoangiography acoustic methods which employ backscatter analysis of transmitted sound waves or measurement of endogenous sound waves for determination of blood vessel characteristics, respectively.

Direct measurement of vessel wall dimensions and/or composition may also be achieved by use of high frequency radiowave measurement, e.g. about 100 GHz or higher, utilizing energy transmitting and receiving structures positioned about vasculature or implanted medical devices. Corresponding control circuitry, power and communication capabilities are understood to be required for this approach and may be accomplished using approaches similar to those utilized in prior embodiments of the invention. Use of high frequency sensors may be extended to include medical devices implanted elsewhere in the body such as in soft tissues, organs, or bone.

As described above with reference to the analyte sensor/drug delivery embodiment, the vascular electrodes described above could also be used to affect or modify the behavior of cells or other substances to reduce foreign body response and/or promote healing and incorporation of the device in the body.

The method and devices of this invention could therefore be used for a multitude of uses and applications, including but not limited to:
Controlling the timing and/or the amount of delivered therapeutic agent to maximize therapeutic effectiveness
Alerting a clinician and/or user to replace an implanted sensor or drug delivery device; in certain embodiments, this alert may precede replacement need, thereby allowing a period in which corrective action may be taken prior to device failure
Providing device design and/or body site guidance based upon feedback of encapsulation reports from one or more users as to the effective lifetime of device performance in-body.
Monitoring for development of stenosis in vascular grafts such that effective therapeutic action may be taken by clinician prior to occlusion or thrombotic activity, thereby extending useful graft lifetime.
Placement of one or more monitoring systems about targeted organs or vascular to gauge foreign body formation and progression to alert clinicians such that effective actions may be taken. Such actions may include a change of therapeutic regimen including use of drugs such as anti-inflammatory agents may be administered or the use of appropriate surgical procedures to remove or repair the foreign body response.

The foregoing description details certain embodiments of the invention. It will be appreciated, however, that no matter how detailed the foregoing appears in text, the invention may be practiced in many ways. It should be noted that the use of particular terminology when describing certain features or aspects of the invention should not be taken to imply that the terminology is being re-defined herein to be restricted to including any specific characteristics of the features or aspects of the invention with which that terminology is associated. Furthermore, while the above detailed description has shown, described, and pointed out novel features of the invention as applied to various embodiments, it will be understood that various omissions, substitutions, and changes in the form and details of the device or process illustrated may be made by those skilled in the technology without departing from the spirit of the invention. The scope of the invention is indicated by the appended claims rather than by the foregoing description. All changes which come within the meaning and range of equivalency of the claims are to be embraced within their scope.

What is claimed is:

1. A method of determining foreign body response about a portion or region of an implanted medical device comprising:
implanting a sensor configured to detect foreign body response;
taking a plurality of measurements using said sensor; and
communicating said sensor measurements to allow evaluation of foreign body formation over time thereby facilitating subsequent therapeutic intervention.

2. The method of claim 1, wherein said sensor is incorporated into or provided on an implanted or partially implanted medical device.

3. The method of claim 2, wherein said medical device comprises an analyte sensor or drug delivery port.

4. The method of claim 1, wherein said sensor is implanted on or in a luminal vascular structure.

5. The method of claim 1, wherein said sensor is implanted on or near a vascular graft.

6. The method of claim 5, wherein said sensor is implanted proximate to an anastomosis.

7. A method of determining analyte concentration in an implanted device, said method comprising:
sensing at least one analyte concentration with an analyte sensor in said implanted device;
sensing encapsulation of said implanted device while said implanted device remains implanted; and
evaluating accuracy of sensed analyte concentration based at least in part on the presence or absence of sensed encapsulation.

8. The method of claim 7, wherein sensing encapsulation comprises sensing impedance between a pair of electrodes.

9. A medical device having an implantable portion comprising:
at least one encapsulation sensor; and
transmission circuitry coupled to said sensor and configured to transmit sensor data for evaluation and possible therapeutic intervention.

10. The medical device of claim 9, comprising a power source.

11. The medical device of claim 9, comprising at least one of an analyte sensor or a fluid delivery port.

12. The medical device of claim 9, wherein said encapsulation sensor comprises an impedance sensor.

13. The medical device of claim 12, comprising at least two electrodes, at least one of which is positioned proximate to said analyte sensor and/or fluid delivery port.

14. The medical device of claim 9, wherein said encapsulation sensor comprises a sonic transmitter and receiver.

15. The medical device of claim 9, wherein said encapsulation sensor comprises a high frequency radiowave transmitter and receiver.

16. A medical device having an implantable portion comprising:
at least one portion resulting in a foreign body response; and
at least one electrode positioned proximate vicinity of said foreign body response;
at least one additional electrode; and
an electrical signal generator connected across at least two of said electrodes and configured to cause current to pass between said at least two electrodes; and
a sensing circuit configured to measure electrical impedance between said at least two electrodes.

17. The medical device of claim 16, comprising a vascular graft.

18. The medical device of claim 16, comprising transmission circuitry coupled to said sensor and configured to transmit sensor data for evaluation and possible therapeutic intervention.

19. The medical device of claim 16, comprising one or both of an analyte sensor and an fluid delivery port.

20. A method of treating a subject with an implanted medical device, said method comprising:
detecting encapsulation of said implanted device while said implanted device remains implanted; and
replacing said implanted device when adverse encapsulation is detected.

21. The method of claim 20, comprising acquiring sensor data indicative of encapsulation.

22. The method of claim 20, comprising transmitting said sensor data or data derived from said sensor data from said implanted device.

23. The method of claim 20, wherein said detecting comprises measuring electrical impedance.
